(19) United States
(12) Patent Application Publication
(10) Pub. No.: US 2010/0152452 A1
(43) Pub. Date: Jun. 17, 2010

(54) STEREOSELECTIVE SYNTHESIS OF PIPERIDINE DERIVATIVES

(75) Inventors: Shan-Yen Chou, Taipei City (TW); Chi-Hsin Richard King, Holladay, UT (US)

Correspondence Address: OCCHIUTI ROHLICEK & TSAO, LLP, 10 FAWCETT STREET, CAMBRIDGE, MA, 02138, US

(73) Assignee: TaiGen Biotechnology Co., Ltd., Taipei City (TW)

(21) Appl. No.: 12/636,956

(22) Filed: Dec. 14, 2009

Related U.S. Application Data
(60) Provisional application No. 61/122,461, filed on Dec. 15, 2008.

(51) Int. Cl. C07D 215/56; C07C 229/24; C07D 211/72; C07C 255/04; C07C 233/04

(52) U.S. Cl. 546/156; 560/171; 546/244; 558/454; 564/160

(57) ABSTRACT

This invention relates to dialdehyde or dinitrile compounds, which are useful for stereoselective synthesis of piperidine, pyrrolidine, and azepane derivatives.

### CROSS-REFERENCE TO RELATED APPLICATIONS

This application claims priority to U.S. Provisional Application No. 61/122,461, filed Dec. 15, 2008. The content of the prior application is hereby incorporated by reference in its entirety.

### BACKGROUND

Piperidine is a six-membered cyclic compound containing five carbon atoms and one nitrogen atom. Its derivatives are widely used as building blocks in the synthesis of piperidine-containing organic compounds for pharmaceutical and other uses.

The stereochemical configurations at the ring atoms of piperidine can be critical to pharmaceutical activity of the piperidine-containing organic compounds. Thus, effectively and stereoselectively synthesizing piperidine derivatives is of great importance.

### SUMMARY

One aspect of this invention relates to dialdehyde or dinitrile compounds, which are useful in making stereochemically pure piperidine derivatives. The compounds of this invention have formula I:

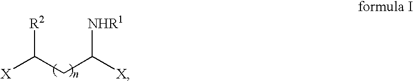

in which R¹ is an amino-protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; X is C(O)H or CN; and n is 0, 1, or 2. The compounds may feature that R¹ is C(O)Ot-Bu, C(O)OCH₂Ph, C(O)CH₃, C(O)CF₃, CH₂Ph, or C(O)O-Ph; or R² is C₁-C₆ alkyl (e.g., methyl).

Referring to the above formula, some of the compounds have the stereochemistry of

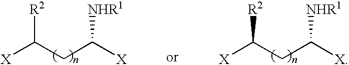

Shown below are two exemplary compounds of this invention:

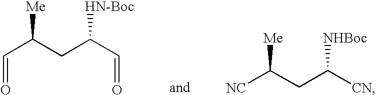

wherein Boc represents t-butoxylcarbonyl.

Another aspect of this invention relates to a synthetic process including contacting the dialdehyde or dinitrile compound of formula I with a compound of formula II:

H₂NR³ formula II,

in which R³ is H, C₁-C₆ alkyl, C₂-C₆ alkenyl, C₂-C₆ alkynyl, C₃-C₈ cycloalkyl, C₁-C₇ heterocycloalkyl, aryl, or heteroaryl, to prepare a piperidine compound of formula III:

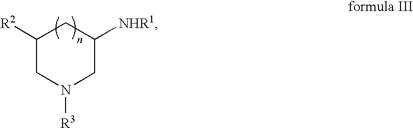

in which R¹, R², R³, and n are as defined above. In one embodiment, R¹ is C(O)Ot-Bu, C(O)OCH₂Ph, C(O)CH₃, C(O)CF₃, CH₂Ph, or C(O)O-Ph; R² is H or C₁-C₆ alkyl (e.g., CH₃); R³ is H or CH₂Ph; and n is 0, 1, or 2.

This process can further include removing R³ from the compound of formula III, wherein n is 1, and coupling the resultant compound with a quinolinone compound to form a compound of the following formula:

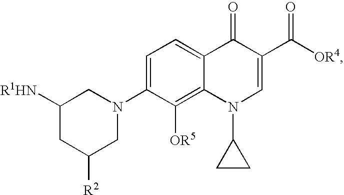

wherein R¹ is H, C(O)Ot-Bu, C(O)OCH₂Ph, C(O)CH₃, C(O)CF₃, CH₂Ph, or C(O)O-Ph; R² is H or C₁-C₆ alkyl; R³ is H or CH₂Ph; R⁴ is H or carboxyl protecting group; and R⁵ is H, C₁-C₆ alkyl, C₂-C₆ alkenyl, C₂-C₆ alkynyl, C₃-C₈ cycloalkyl, C₁-C₇ heterocycloalkyl, aryl, or heteroaryl. The resultant compound may have the following stereochemistry:

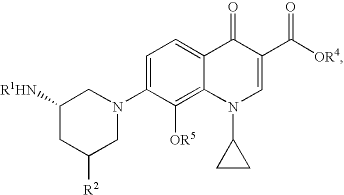

or preferably

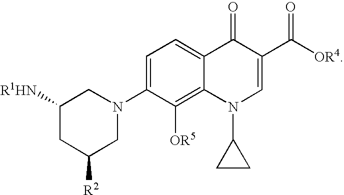

The dialdehyde compound used to prepare the compound of formula (III) can be obtained by conducting a reduction reaction of a diester compound of the following formula

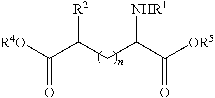

or by reducing the diester compound to a dialcohol compound and then oxidizing the dialcohol compound.

In the above process, when the dialdehyde compound of formula I is

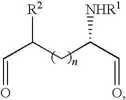

the compound of formula III thus obtained is

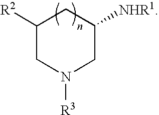

The dialdehyde compound can be obtained by reduction of

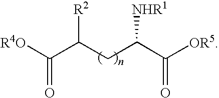

Further, in the above process, when the dialdehyde compound of formula I is

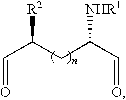

the compound of formula III thus obtained is

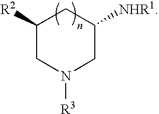

The dialdehyde compound can be obtained by reduction of

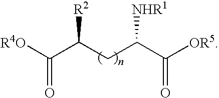

The dinitrile compound used in the above process can be prepared by treating, with a dehydrating agent, a diamide compound of the following formula

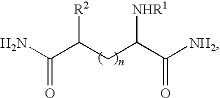

in which R¹ is an amino protecting group; and R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl. The diamide compound can be prepared by direct amidation of the diester compound shown above with ammonia or by hydrolyzing the diester to diacid and subsequent amidation of the diacid.

In the above process, when the dinitrile compound of formula I

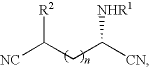

is the compound of formula III thus obtained is

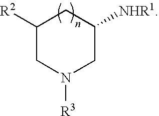

The dinitrile compound can be synthesized by dehydration of

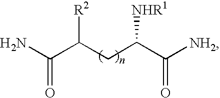

which, in turn, can be prepared by amidation of

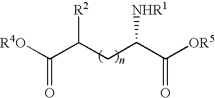

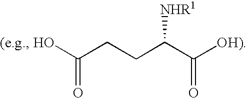

Further, in the above process, when the dinitrile compound of formula I is

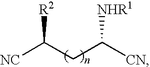

the compound of formula III thus obtained is

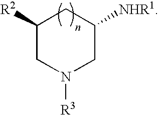

The dinitrile compound can be synthesized by dehydration of

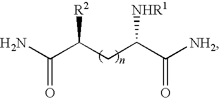

which, in turn, can be prepared by amidation of

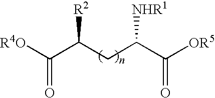

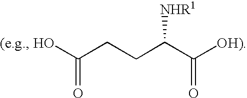

This process can also include treating the following compound:

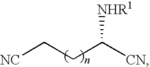

in the presence of a base, e.g., liithium hexamethyldisilazide (LiHDMS), with R²L, wherein R² is alkyl, e.g., methyl, and L is I, Br, MeSO₄; to stereoselectively synthesize the compound of formula I. Further, it may include reacting the compound of formula III, wherein R³ is H, with an acid (e.g., oxalic acid or a chiral acid) to form a salt and stereoselectively purifying the salt.

The term “alkyl” refers to a straight or branched hydrocarbon, containing 1-6 carbon atoms. Examples of alkyl groups include, but are not limited to, methyl, ethyl, n-propyl, i-propyl, n-butyl, i-butyl, and t-butyl. The term “alkoxy” refers to an O-alkyl radical. Examples of alkoxy groups include, but are not limited to, methoxy, ethoxy, and butoxy. The term “alkylene” refers to an alkyl diradical group. Examples of “alkylene” include, but are not limited to, methylene and ethylene.

The term “alkenyl” refers to a straight or branched hydrocarbon having one or more C═C double bonds. Examples of alkenyl groups include, but are not limited to, ethenyl, 1-butenyl, and 2-butenyl.

The term “alkynyl” herein refers to a C₂₋₁₀ straight or branched hydrocarbon, containing one or more C≡C triple bonds. Examples of an alkynyl group include, but are not limited to, ethynyl, 2-propynyl, and 2-butynyl.

The term “aryl” refers to a 6-carbon monocyclic, 10-carbon bicyclic, 14-carbon tricyclic aromatic ring system wherein each ring may have 1 to 4 substituents. Examples of an aryl group include, but are not limited to, phenyl, naphthyl, and anthracenyl. The term “cycloalkyl” refers to a saturated and partially unsaturated cyclic hydrocarbon group having 3 to 12 carbons. Examples of a cycloalkyl group include, but are not limited to, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having one or more heteroatoms (such as O, N, or S). Examples of a heteroaryl group include pyridyl, furyl, imidazolyl, indolyl, indazolyl, benzimidazolyl, pyrimidinyl, thienyl, quinolinyl, and thiazolyl. The term “heteroaralkyl” refers to an alkyl group substituted with a heteroaryl group.

The term “heterocycloalkyl” refers to a nonaromatic 3-8 membered monocyclic, 8-12 membered bicyclic, or 11-14 membered tricyclic ring system having one or more heteroatoms (such as O, N, or S). Examples of a heterocycloalkyl group include, but are not limited to, piperazinyl, pyrrolidinyl, dioxanyl, morpholinyl, and tetrahydrofuranyl. Heterocycloalkyl can be a saccharide ring, e.g., glucosyl.

Alkyl, alkenyl, alkynyl, cycloalkyl, heterocycloalkyl, aryl, and heteroaryl mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, and heterocyclyl, in which the alkyl, alkenyl, alkynyl, alkyloxy, aryl, heteroaryl, cyclyl, and heterocyclyl can be further substituted.

The term “amino protecting group” refers to a functional group that, when bonded to an amino group, prevents the amino group from interference. This protecting group can be removed by conventional methods. Examples of amino protecting groups include, but are not limited to, alkyl, acyl, and silyl. Commonly used amino protecting groups are C(O)Ot-Bu, C(O)OCH₂Ph, C(O)CH₃, C(O)CF₃, CH₂Ph, and C(O)O-Ph. Amino protecting groups have been discussed in T. W. Greene and P. G. M. Wuts, Protective Groups in Organic Synthesis, 2d. Ed., John Wiley and Sons (1991).

The term “dehydrating agent” refers to a chemical agent that, upon contacting another chemical substance, removes water from that substance. Examples of a dehydrating agent include, but are not limited to, benzenesulfonyl chloride, cyanuric chloride, ethyl dichlorophosphate, phosphorus oxychloride, or phosphorus pentoxide.

Other features, objects, and advantages of the invention will be apparent from the description and the claims.

### DETAILED DESCRIPTION

The dialdehyde compounds of this invention can be prepared by well-known methods. For example, as illustrated in Scheme 1 below, a dialdehyde compound can be prepared from commercially available L-glutamic acid. More specifically, one can protect the amino and carboxyl groups of diacid 1 to obtain compound 2, and then conduct alkylation of compound 2, with a alkykating agent, such as MeI, MeBr, and Me₂SO₄, to form compound 3. Note that the stereoselectivity of alkylation of compound 2 at the C-4 position can be controlled by the stereochemistry of the C-2 position. Thus, the 4S isomer of compound 3 is predominantly formed. See Hanessian et al., Tetrahedron Lett., 1998, 39, 5887; and Gerwick et al., Tetrahedron Lett., 2003, 44, 285. After this stereoselective alkylation, compound 3 is reduced to give desired dialdehyde compound 4, in which the stereochemistry of the C-2 and C-4 positions is maintained.

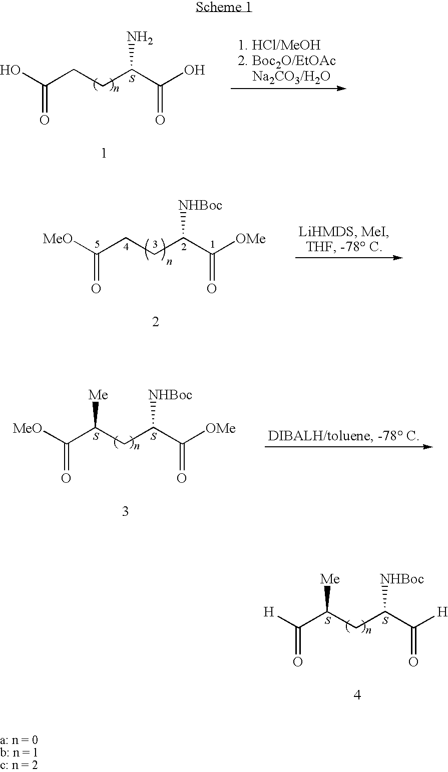

The dialdehyde compounds described herein can be reacted with a primary amine or ammonia under reductive amination condition, which requires a reducing agent, to form a piperidine compound. Reducing agents used in reductive amination are well known in the art. Examples include NaBH₄, NaCNBH₃, and NaBH(OAc)₃. As shown in Scheme 2 below, dialdehyde compound 4 is reacted with benzylamine and NaBH₄ to form N-benzyl piperidine compound 5 and reacted with ammonia and NaBH₄ to form N-free cyclic N-containing compound 6:

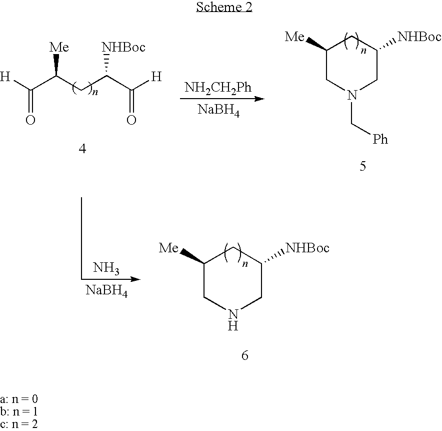

Dialdehyde compounds may be unstable and can be used for a further reaction without isolation or purification. Scheme 3 below depicts a one-pot process of converting protected L-glutamic acid 2b to piperidine compound 6b, which is reacted with oxalic acid to give piperidine oxalate compound 7b. In this process, the intermediates dialdehyde compound 4b is not isolated from the reaction.

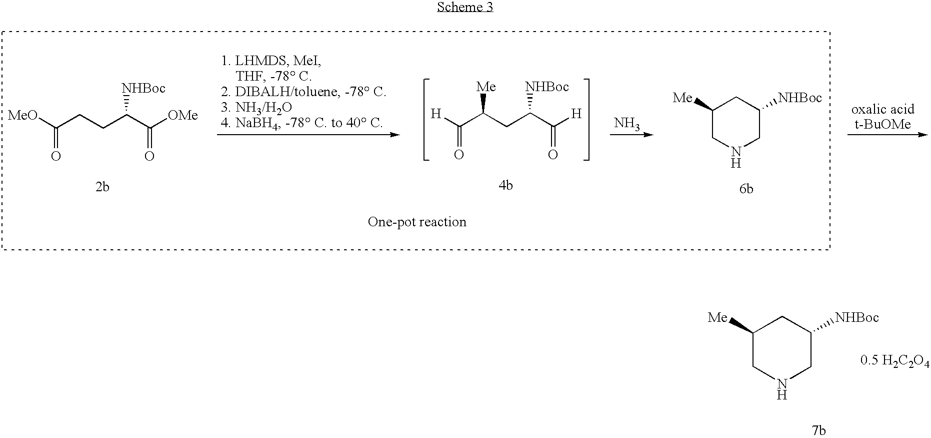

Shown below are some other piperidine compounds and enantiomers that can be prepared from dialdehyde compounds.

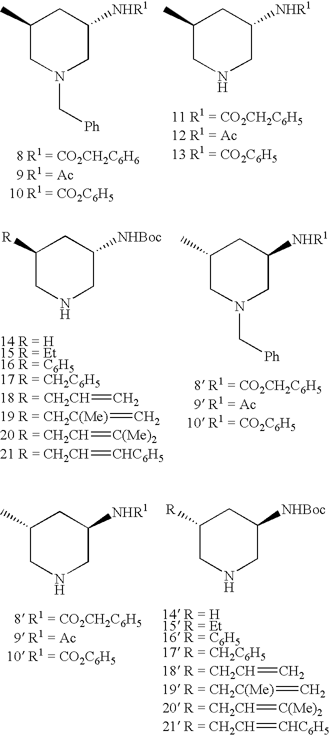

The piperidine compound can be used as a building block for synthesizing other organic compounds.

The dialdehyde compounds described above can be also prepared from diester by a reduction-oxidation sequence. For example, as illustrated in Scheme 4 below, diester compound 3 is reduced in the presence of LiAlH₄ to form a dialcohol compound 22, which was subjected to Swern oxidation to afford dialdehyde compound 4:

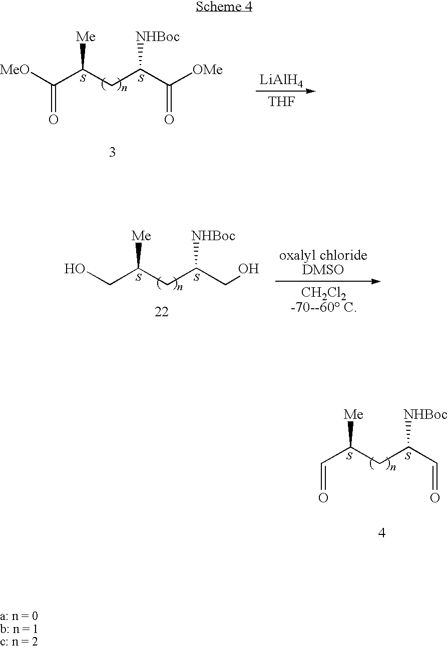

Like the dialdehyde compounds, the dinitrile compounds, available by dehydrating the corresponding diamides, can be used to prepare cyclic N-containing compounds. For example, as illustrated in Scheme 5 below, the diester compound is subjected to amination to afford diamide compound 23, which is treated with a dehydrating agent to give dinitrile compound 24. Dinitrile compound 24 is then reacted with ammonia or benzylamine in one-pot under catalytic hydrogenation condition to give compound 6:

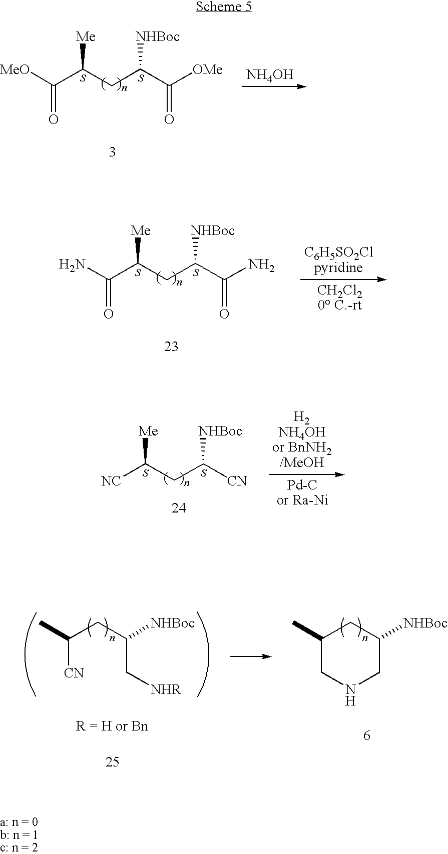

Resolution of compound 6 can be achieved by reacting it with an acid (e.g., oxalic acid) to give its salt form, followed by crystallization or trituration using appropriate solvent systems. In certain instances, a chiral acid may be used. The diastereomeric excess (de) value of thus-purified compound 6 can exceed 99.9%.

Scheme 6 below shows an alternative one-pot process to synthesize diamide 23 used to prepare piperdine compounds as demonstrated in Scheme 5. Diester compound 3 is hydrolyzed to give diacid compound 26, which is subjected to amination under a mild condition to afford diamide 23. See Pozdnev, V. F. Tetrahedron Letters, 1995, 36, 7115. This method minimizes the possibility of racemization, as it requires a mild condition.

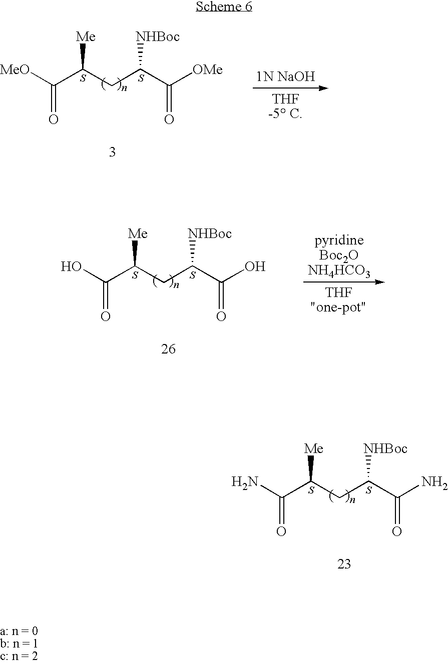

Diacid 26b can also be prepared by alkylation of y-methyl-N-Boc-L-glutamate in the presence of lithium diisopropylamide, followed by hydrolysis of the intermediates (26b′, 26b″), as shown in Scheme 7 below. The diastereomeric excess (de) value of the alkylation product 26b, determined by HPLC analysis of diamide 23 obtained from 26b, is very high.

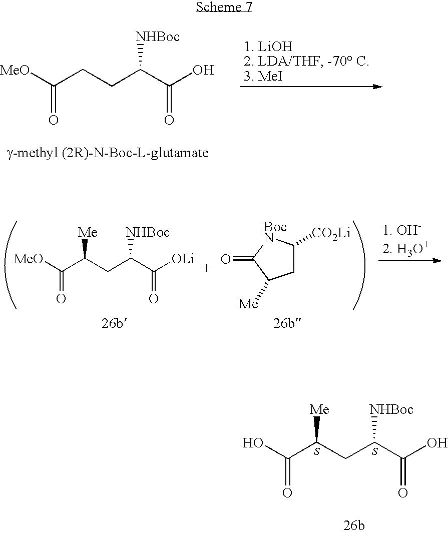

Diamide 23 can be converted into dinitrile 24 at low temperature using cyanuric chloride as a dehydration agent. See Scheme 8 below. This dehydration method is described in Aureggi, V. et. al. Org. Synth. 2008, 85, 72.

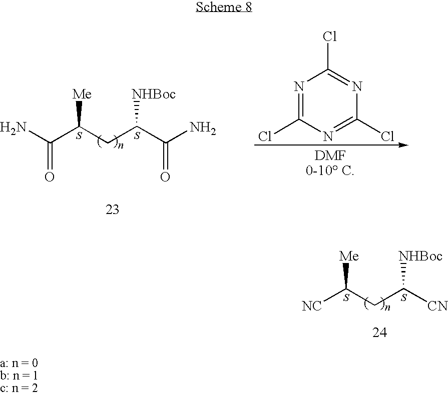

Alternatively, dinitrile 24 can be synthesized from diacid 26 in a one-pot fashion as illustrated in Scheme 9 below.

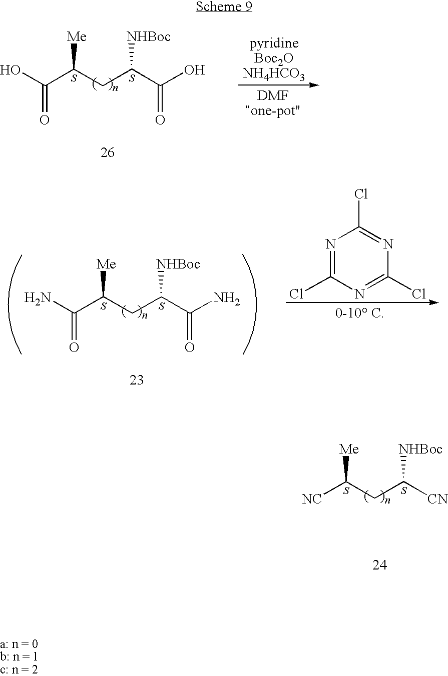

Shown in Scheme 10 below is a synthetic approach to piperidine 6b from commercially available L-glutamic acid:

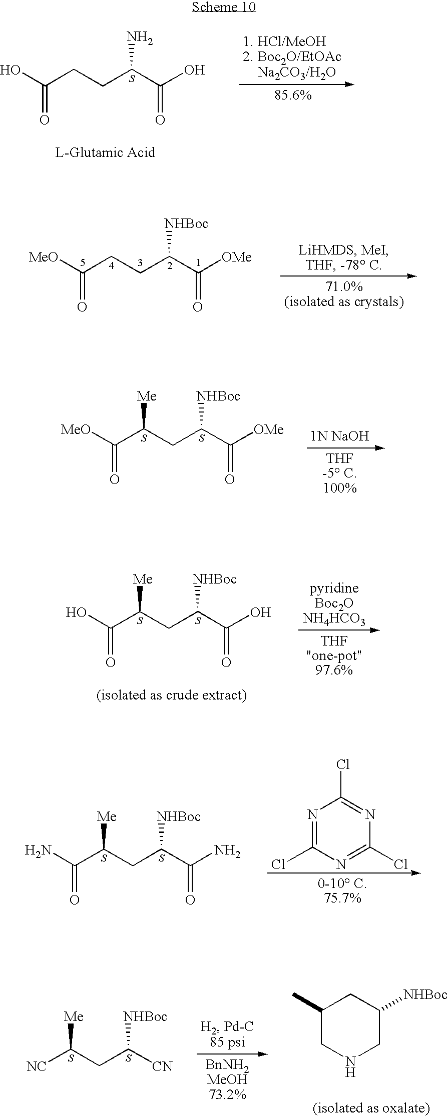

Scheme 11 below shows another synthetic approach to piperidine 6b:

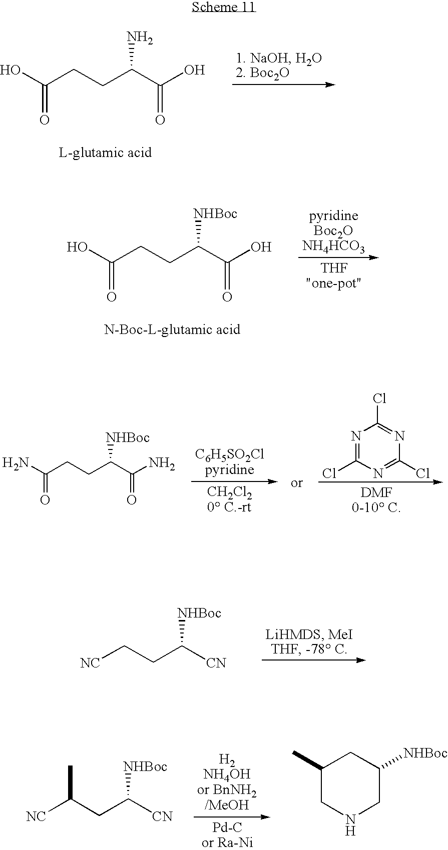

The method described above can also be used to synthesize pyrrolidine and azepane under mild conditions. Shown in Scheme 12 below is a general synthetic route to 5-7 membered cyclic N-containing compounds:

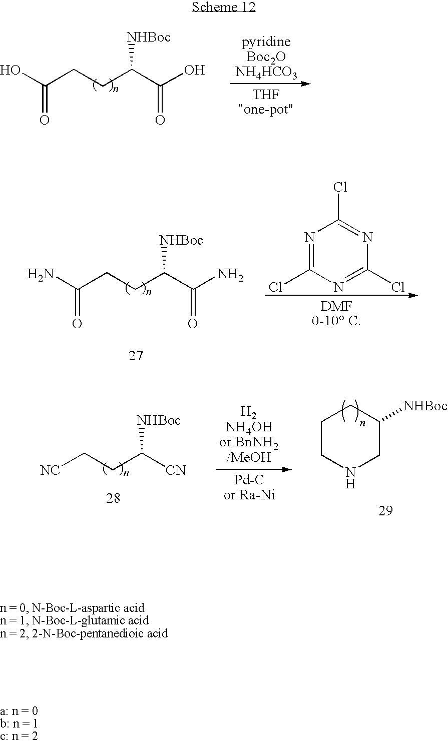

The cyclic N-containing compounds are useful building blocks for synthesizing other organic compounds. (35)-3-(tent-butoxycarbonylamino)-pyrrolidine (compound 29a) can be used to synthesize Rho-kinase inhibitors. See PCT publications WO 2008105442 and WO 2008105058. (35)-3-(tert-butoxycarbonylamino)-piperidine (compound 29b) can be used to synthesize Tie-2-kinase inhibitors. See J. Med. Chem., 50, 2007, 627-640). The antipode of compound 29b, (3R)-3-(tert-butoxycarbonylamino)-piperidine, has been widely used to produce dipeptidyl peptidase IV (DPP-4) inhibitors such as Alogliptin. See PCT publication WO 2007112368. 3-tert-Butoxycarbonylaminohexahydro-2-azepine (compound 29c) can be used to synthesize CHK1 inhibitors and DPP-4 inhibitors. See PCT publications WO2005066163 and WO2002068420.

Scheme 13 below shows that piperidine compound 6b is coupled with quinolinone 30 to form intermediate 31, which, after undergoing deprotection and acidification, affords compound 34, an antibacterial drug candidate (see U.S. Pat. No. 6,329,391):

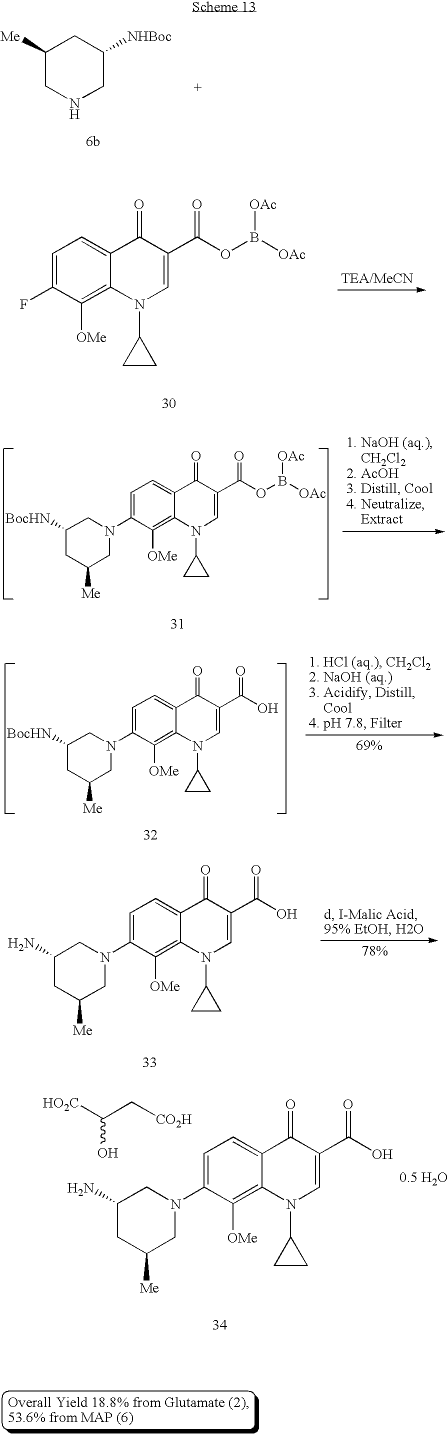

Schemes 1-13 shown above are merely illustrative. Modifications can be made to prepare and use the compounds of invention. Chemical transformations useful in practicing this invention can be found, for example, in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Example 1

### Synthesis of (S)-2-tert-butoxycarbonylamino-pentanedioic acid dimethyl ester (compound 2b)

L-Glutamic acid (200 g) and MeOH (800 mL) were charged into a three-liter four-necked flask and then cooled to −10° C. SO₂Cl₂ (324 g) was added dropwise at <10° C. and the mixture was stirred at room temperature for 18 hours. The reaction was monitored by LC/MS. Ethyl acetate (800 mL), Na₂CO₃ (200 g), H₂O (200 g), and di-tert-butyldicarbonate (280 g) were sequentially added. After stirring for 18 hours at room temperature, the resulting mixture was washed with water (400 mL×2) and then diluted with toluene (400 mL). The organic layer was separated and evaporated under vacuum to give compound 2b (314 g, 84% crude yield).

### Example 2

### Synthesis of (2S,45)-2-tent-butoxycarbonylamino-4-methyl-pentanedioic acid dimethyl ester (compound 3b)

1 M LiHMDS in THF (1500 mL) was charged into a five-liter four-necked flask at −78° C. under nitrogen. To this was added dropwise a solution containing crude compound 2b (210 g in 1000 mL dry THF) at <−60° C., and then stirred for 1.5 h at −78° C.

To the resulting solution was added MeI (175 g in 100 mL dry THF) at <−60° C. The reaction was stirred at −78° C. for 4 h and then quenched with MeOH (35 g) at −60° C. and 2 N HCl (1500 mL) at −10° C. To the resulting solution was added toluene (1000 mL) and stirred for 0.5 h. The organic layer was separated and treated with a Na₂S₂O₃ solution (175 g in 1000 ml, water) with stirring for 30 minutes, during which period, the color of the solution turned from dark brown to pale yellow. The organic layer was evaporated under vacuum to give compound 3b (212 g, 96% crude yield). ¹H NMR (CDCl₃, 300 MHz): δ1.22 (d, J=6.9 Hz, 3H), 1.43 (s, 9H), 1.45 (m, 1H), 1.86 (ddd, 1H), 2.00 (dd, 2H), 2.58 (dd, 1H), 3.67 (s, 3H), 3.73 (s, 3H), 4.35 (br s, 3H), 4.97 (d, J=6.0 Hz, 1H); MS: m/e 312.0 (M⁺+23).

### Example 3

### One-pot synthesis of (3S,55)-3-(tert-butoxycarbonylamino)-5-methyl-N-benzyl-piperidine (compound 5b)

A solution of the crude compound 3b (50.0 g) in toluene (750 mL) was cooled to −78° C. with stirring under nitrogen. To the solution was added dropwise cold DIBALH (500 mL, 1 M in toluene, −78° C.) at <−60° C. to give (2S,45)-2-tert-butoxycarbonylamino-4-methyl-pentanedialdehyde (i.e., compound 4b). After stirring for 30 minutes at −78° C., a mixture of benzylamine (22.5 g in 25 mL of toluene) and MeOH (12.5 mL) was added. The cooling bath was removed to allow the solution temperature to rise to −10° C. NaBH₄ (6.5 g) and acetic acid (10.0 g) were then added. After the reaction mixture was stirred at room temperature for 18 h, it was treated with 2 N HCl (3000 mL) at −10° C. The aqueous layer was subjected to extraction with dichloromethane (500 mL×3). The combined organic layers were concentrated to give brown oil, which was purified by a short pad of silica gel eluted with ethyl acetate, 1/4 (v/v) methanol/ethyl acetate, and 4/16/80 (v/v/v) ammonia water/methanol/ethyl acetate to afford compound 5b (15.6 g, 30%). ¹H NMR (CDCl₃, 300 MHz): δ 0.83 (d, J=7.0 Hz, 3H), 1.04 (ddd, 1H), 1.45 (s, 9H), 1.55 (ddd, 1H), 1.79-1.81 (m, 2H), 2.12 (dd, 1H), 2.67-2.71 (m, 2H), 3.43 (d, 1H), 3.46 (d, 1H), 3.85 (m, 1H), 5.33 (d, 1H), 7.22-7.42 (m, 5H); MS: m/e 305.0 (M⁺+1).

Alternatively, Compound 5b was prepared by the following methods.

A solution of the crude 3b (38.0 g) in toluene (650 mL) was cooled to −78° C. with stirring under nitrogen. To the solution was added dropwise cold DIBALH (700 mL, 1 M in toluene, 78° C.) at <60° C. After stirring for 30 minutes at −78° C., a solution of benzylamine (15.0 g in 45 mL of MeOH) was added. Then the cooling bath was removed to allow the solution temperature rise to −10° C. NaCNBH₃ (15.0 g) and ethyl acetate (300 mL) were then added. After the reaction mixture was stirred at room temperature for 18 hours, it was treated with 2 N HCl (700 mL) at −10° C. The aqueous layer was subjected to extraction with dichloromethane (200 mL×2). The combined organic layers were concentrated to give brown oil, which was purified by a short pad of silica gel and eluted with ethyl acetate, methanol/ethyl acetate 1:4 (v/v) and ammonia water (28-30%)/methanol/ethyl acetate 4/16/80 (v/v/v) to afford compound 5b (10.0 g, 25.0%).

Compound 5b was converted to compound 5.HCl as follows:

A suspension of compound 5b in toluene was titrated with HCl in ether (1M) to equivalent point at 0-5° C. The resulting solution formed crystals on standing. The crystals were collected by filtration, washed with tert-BuOMe, and dried to give compound 5.HCl (9.8 g, 100%) as a white powder. Mp: 173° C. (toluene).

### Example 4

### Synthesis of (3S,5S)-3-(tent-butoxycarbonylamino)-5-methylpiperidine hydrogen chloride (compound 6b.HCl)

Compound 5b.HCl (3.3 g) was dissolved in methanol (100 mL). To this was added 10% Pd—C catalyst (0.74 g). The solution was stirred in a Parr hydrogenation flask for 24 hours under H₂ at the pressure of 75 psi. After filtering off the catalyst, the volatiles were removed under reduced pressure to give yellow oil, which was triturated with diethyl ether. The resulting solution gave precipitates on stirring. The precipitates were collected by filtration, washed with tert-BuOMe, and dried to give compound 6b.HCl (2.5 g, 96.6% purity) as a white powder. Mp: 168° C. (diethyl ether).

### Example 5

### One-pot synthesis of (3S,55)-3-(tert-butoxycarbonylamino)-5-methylpiperidine (compound 6b)

A solution of the crude compound 2b (16.0 g) in toluene (240 mL) was cooled to −78° C. with stirring under nitrogen. To the solution was added dropwise cold DIBALH (160 mL, 1 M in toluene) at such a rate that the solution temperature remained at <−60° C. After stirring for 1 hour at −78° C., ammonia aqueous solution (50 mL, 30%) and acetic acid (1.7 g) were added. The cooling bath was replaced with an ice bath and the reaction mixture was stirred at 0-5° C. for 1.5 hours. NaBH₄ (1.1 g) was added and the reaction was monitored by LC/MS. After 1 h, additional NaBH₄ (0.5 g) was added. The ice bath was removed and the reaction was stirred at room temperature for 18 hours. Celite (45 g) was added with stirring. The mixture was heated to 50° C. to remove ammonia and filtered through Celite-alumina gel in a sintered glass funnel with suction. The filtrate was subjected to extraction with 10% KHSO₄ aqueous solution (80 mL×2). The Celite-alumina gel was rinsed 3 times with 185 mL of 1/10 (v/v) methanol/ethyl acetate for more than 10 minutes and then filtered with suction. The combined filtrates were subjected to extraction with 10% KHSO₄ aqueous solution (100 mL x 2). All of the KHSO₄ extracts were combined, washed with toluene (50 mL×2), neutralized with ammonia, and extracted with ethyl acetate (200 mL×3). The combined organic layers were evaporated under vacuum to give crude compound 6b (7.3 g, 61%). An analytical sample was prepared by silica gel column chromatography purification using 1/10/0.05 (v/v/v) methanol/ethyl acetate/ammonia water and followed by crystallization from hexane to give compound 6b as pale yellow granular crystal. Mp: 63-64° C. (hexane); ¹H NMR (CDCl₃, 300 MHz): δ 0.80 (d, J=6.6 Hz, 3H), 1.14 (ddd, 1H), 1.40 (s, 9H), 1.58 (ddd, 1H), 1.95 (dd, 1H), 2.16 (m, 1H), 2.65 (dd, 1H), 2.82 (dd, 1H), 2.90 (dd,1H), 3.70 (m, 1H), 5.41 (m,1H); MS: m/e 215.2 (M⁺+1).

Compound 6b was also prepared in the scales of 50 gram and 100 gram in the manners similar to that described above.

Resolution of compound 6b was achieved by converting it to the salt form followed by crystallization or trituration using appropriate solvent systems. Table 1 below shows that resolution with various acids and recyrstalliaztion/trituration with various solvents afford high diastereomeric excess (de) values.

TABLE 1
Purification of compound 6b in salt formation

| | | | | de | | |
|---|---|---|---|---|---|---|
| | | | | value | | |
| | Resolving | | | (%), | Yield | de value |
| | acid (0.5 | Solvent | | crude | (free | (%), |
| Entry | molar eq.) | system | Method | 6b | base) | pure 6b |
| 1 | d-tartaric | acetone/ | recrystallization | 95 | 69 | 99.8 |
| | acid | water | | | | |
| | | 18/1 (v/v) | | | | |
| 2 | di-o- | acetone | recrystallization | 95 | 78 | 98.8 |
| | toluoyl-d- | | | | | |
| | tartaric | | | | | |
| | acid | | | | | |
| 3 | oxalic | i-PrOH/ | recrystallization | 95 | 70 | 98.2 |
| | acid | water | | | | |
| | | 10/1 (v/v) | | | | |
| 4 | oxalic | acetone | hot trituration | 97.5 | 71 | 99.0 |
| | acid | | | | | |
| 5 | oxalic | acetone/ | hot trituration | 97.5 | 73 | >99.9 |
| | acid | water | | | | |
| | | 20/1 (v/v) | | | | |

### Example 6

### Synthesis of (3S, 5S)-3-(tert-butoxycarbonylamino)-5-methylpiperidine oxalate (compound 6b•0.5 H₂C₂O₄)

Crude compound 6b (7.3 g) and a saturated solution of oxalic acid (1.5 g) in methanol were suspended in tert-BuOMe (100 mL) at 40° C. The mixture was cooled to room temperature and stirred for 48 hours. Precipitates were formed while stirring. The precipitates were collected by filtration, washed with tert-BuOMe, and dried to provide compound 6b•0.5 H₂C₂O₄ (7.3 g, 83% recovery, >97.0% purity) as a white powder. Mp: 203° C. (tert-BuOMe). Recrystallization of the crude oxalate from 1/4 (v/v) methanol/tert-BuOMe gave pure compound 6b•0.5 H₂C₂O₄ in the recovery rate of 82%.

Compound 6 was obtained as white powders, Mp: 63-64° C. (hexane), by treating compound 6b•0.5 H₂C₂O₄ with a base. Its NMR data was identical to that compound 6b previously prepared.

### Example 7

### One-pot synthesis of (3S,55)-3-(tent-butoxycarbonylamino)-5-methylpiperidine oxalate (compound 6b•0.5 H₂C₂O₄)

LiHMDS solution (520 mL of 1 M in THF) was charged into a one-liter four-necked flask at −78° C. under nitrogen. To this solution was added dropwise at <−60° C. a solution of crude Compound 2b (60.0 g in 300 mL of dry THF). The reaction mixture was stirred for 1.5 h at −78° C. MeI (44.4 g in 20 mL dry THF) was added. After stirring for 2 h at −70° C., diisopropylamine (30.0 g) was added to quench unreacted MeI. The mixture was stirred at −70° C. for 2.5 h.

To the solution was added dropwise cold DIBALH (600 mL, 1 M in toluene) at such a rate that the solution temperature remained <−60° C. (˜one hour period). After stirring for 0.5 h at −70° C., ammonia aqueous solution (360 mL, 30%) was added to the mixture within a five-minute period. The reaction temperature was allowed to rise to −40° C., ammonia gas (˜70-80 g) was introduced, followed by addition of NaBH₄ (12.0 g). After stirring at −10° C. for 10 hours, the reaction temperature was allowed to further rise to room temperature during a six-hour period while monitored by LC/MS. The released ammonia was trapped by ice water. 20% NaOH aqueous solution (400 mL) was added with stirring. The mixture was filtered through alumina gel in a sintered glass funnel with suction. The organic layer of the filtrate was washed with water (300 mL×2), and evaporated under vacuum to give crude compound 6b (16.4 g). The alumina gel was thoroughly washed with methanol and filtered with suction. The filtrate was evaporated under vacuum. The residue was then filtered through Celite and washed with ethyl acetate. The filtrate was concentrated under vacuum to give crude compound 6b (5.0 g). The combined crude product was purified by flash column chromatograph eluted with ethyl acetate to 1/10/0.1 (v/v/v) methanol-ethyl acetate-triethylamine to provide pure compound 6b (10.8 g, 23% based on crude compound 2b).

To determine the optical purity of compound 6b, its optical antipode (i.e. (3R,5R)) was synthesized in the same manner as those described in Examples 1-7 except D-glutamic acid was used instead of L-glutamic acid. Both compound 6b and its optical antipode were derivatized with (S)-(+)-1-(1-naphthyl)ethyl isocyanate, and the resulting chiral ureas were subjected to HPLC analysis. The results showed that compound 6b had an optical purity greater than 98%.

### Example 8

### Syntheses of piperidine compounds 8-21

Compounds 8-10 were individually synthesized in the same manner as those described in Examples 1-3 except that amino protecting agents different from di-tert-butyldicarbonate were used.

Compound 8, ¹H NMR (CDCl₃, 300 MHz): δ0.81 (d, J=6.3 Hz, 3H), 1.04 (ddd, 1H), 1.58 (ddd, 1H), 1.84-1.88 (m, 2H), 2.16 (dd, 1H), 2.68-2.78 (m, 2H), 3.43-3.48 (m, 2H), 3.90 (m, 1H), 5.05 (s, 2H), 5.75 (br s, 1H), 7.22-7.42 (m, 10H); MS: m/c 339.2(M⁺+1), compound 8.HCl, white powder: Mp: 215° C. (tert-BuOMe).

Compound 11-13 were synthesized in the same manner as those described in Examples 1 and 5 except that amino protecting agents different from di-tert-butyldicarbonate were used.

Compound 11, ¹H NMR (CDCl₃, 300 MHz): δ0.83 (d, J=6.6 Hz, 3H), 1.19 (ddd, 1H), 1.70 (m, 1H), 1.82 (ddd, 1H), 2.20 (m, 1H), 2.71 (dd, 1H), 2.88 (dd, 1H), 2.95 (dd,1H), 3.83 (m, 1H), 5.10 (s, 2H), 5.62 (m, 1H), 7.25-7.40 (m, 5H); MS: m/e 249.2 (M⁺+1).

Compound 11•0.5 H₂C₂O₄ was prepared as a white powder. Mp: 155° C. (tert-BuOMe).

Compounds 14-21 were synthesized in the same manner as those described in Examples 1 and 5 except that alkylation was either not conducted or conducted with different alkylating agents.

Compound 14: Mp: 120-122° C. (hexane); ¹H NMR (CDCl₃, 300 MHz): δ 4.80-4.87 (m, 1H), 3.45-3.55 (m, 1H), 2.98, 3.02 (ABq, J=3.0 Hz, 1H), 2.73-2.79 (m, 1H), 2.57-2.63 (m, 1H), 2.44-2.50 (m, 1H), 1.75-1.79 (m, 1H), 1.60-1.70 (m, 1H), 1.46-1.55 (m, 1H), 1.44 (s, 9H); MS: m/e 201.2 (M⁺+1).

Compound 15: ¹H NMR (CDCl₃, 300 MHz):δ0.78 (t, 3H), 1.29 (m, 2H), 1.35 (s, 9H), 1.40 (ddd, 1H), 1.73-1.76 (m, 2H), 2.08-2.15 (t, 1H), 2.65 (dd, 1H), 2.82 (dd, 1H), 2.90 (dd,1H), 3.70 (m, 1H); MS: m/e 229.2 (M⁺+1).

Compound 16: ¹H NMR (CDCl₃, 300 MHz):δ1.40 (s, 9H), 1.62 (dd, 1H), 2.20-2.40 (m, 2H), 2.50 (dd, 2H), 2.82 (dd, 1H), 2.90 (dd,1H), 3.75 (m, 1H), 7.13-7.32 (m, 5H); MS: m/e 277.2 (M⁺30 1).

Compound 17: ¹H NMR (CDCl₃, 300 MHz): δ1.40 (s, 9H), 1.58 (ddd, 2H), 2.20-2.40 (m, 3H), 2.50 (m, 2H), 2.65 (dd, 2H), 3.75 (m, 1H), 7.13-7.32 (m, 5H); MS: m/e 291.4 (M⁺+1).

Compound 18: ¹H NMR (CDCl₃, 300 MHz): δ1.40 (s, 9H), 1.81 (s, 1H), 1.91-1.95 (m, 1H), 2.03-2.22 (t, 2H), 2.68-2.72 (d, 2H), 2.84-3.01 (dd, 2H), 3.76 (m, 1H), 4.96 (dd,1H), 4.99-5.01 (m, 1H), 5.68-5.77 (m,1H); MS: m/e 241.2 (M⁺+1).

Compound 19: ¹H NMR (CDCl₃, 300 MHz): δ1.40 (s, 9H), 1.65 (s, 3H), 1.81 (m, 2H), 1.96 (m, 2H), 2.03-2.22 (m, 1H), 2.68-2.72 (d, 2H), 2.84-3.01 (m, 2H), 3.76 (m, 1H), 4.58(s, 1H), 4.68 (s, 1H); MS: m/e 255.2 (M⁺+1).

Compound 20: ¹H NMR (CDCl₃, 300 MHz): δ1.40 (s, 9H), 1.54 (s, 3H), 1.76 (s, 3H), 2.16 (m, 1H), 2.65 (dd, 1H), 2.82 (dd, 1H), 2.68-2.73 (dd, 2H), 2.84-2.88 (d, 2H), 3.00-3.04 (dd, 2H), 3.74 (m, 1H), 5.02-5.07 (m,1H); MS: m/e 269.2 (M⁺+1).

Compound 21: ¹H NMR (CDCl₃, 300 MHz): δ1.40 (s, 9H), 1.58 (ddd, 1H), 1.95 (dd, 1H), 2.16 (m, 1H), 2.68-2.73 (dd, 2H), 2.84-2.88 (d, 2H), 3.00-3.04 (dd, 2H), 3.78 (s, 1H), 6.08-6.32 (m, 1H), 6.32-6.37 (d, 1H), 7.15-7.33 (m, 5H); MS: m/e 317.2 (M⁺+1).

### Example 9

### Synthesis of (3S,5S)-3-(tert-butoxycarbonylamino)-5-methyl-N-benzyl-piperidine (compound 5b) by oxidation-reductive amination sequence

A solution of compound 3b (10.2 g) in THF (50 mL) was added to an ice-cooled suspension of LiAlH₄ (3.8 g) in THF (150 mL) with stirring. After stirring for one hour at room temperature, the reaction was re-cooled to 0° C. and treated with 35 mL of 10% KOH. The resulting mixture was filtered from Celite and evaporated. The residual oil was purified by flash column chromatography using ethyl acetate as the eluent to yield the diol 22b (6.9 g, 84%). ¹H NMR (CDCl₃, 300 MHz): 6 0.94 (d, J=7.0 Hz, 3H), 1.43 (s, 9H), 1.56-1.65 (m, 1H), 1.66-1.83 (m, 2H), 3.38-3.42 (m, 1H), 3.43-3.60 (m, 2H), 3.60-3.70 (m, 1H), 3.71-3.80 (m, 1H), 4.86 (br s, 1H); MS: 256.0 (M⁺+23).

To a cooled dichloromethane (149 mL, −70° C.) was added oxalyl chloride (9.1 g) with stirring. After 5 minutes, dry DMSO (11.2 g) was added dropwise at −65° C. to −70° C. Then to this was added a solution of (2S,4S)-2-tent-butoxycarbonylamino-4-methyl-pentane-1,5-diol 22b (6.9 g) in dichloromethane (35.5 mL). After stirring for 15 minutes at −65° C. to −70° C., a pre-cooled triethylamine (26.5 g, −70° C.) was added, and stirring was continued for 15 minutes. Then the mixture was treated with a solution of Oxone (6.0 g) in water (113 mL) while stirring. The separated organic layer was transferred to a flask, cooled to −50° C., and treated sequentially with anhydrous MgSO₄ (3.1 g) and a pre-cooled solution of benzylamine (3.5 g, −50° C.) in THF (20 mL). After 15 minutes, sodium triacetoxyborohydride (18.8 g) was added and stirred at −15° C. to 0° C. overnight. The resulting mixture was washed with brine and the separated organic layer was evaporated. Purification of the residue by a short pad of silica gel using 1/20 to 1/10 (v/v) ethyl acetate-hexanes yielded compound 5b (4.8 g, 53.3%).

### Example 10

### Synthesis of (2S,45)-2-tent-butoxycarbonylamino-4-methyl-pentanedioic acid diamide (compound 23b)

Method A: A suspension of compound 3b (33.0 g, 114.0 mmol) in ammonia water (28-32%, 300 mL) was stirred at room temperature. The mixture gradually changed from granular yellow powder suspension to white solid suspension within three to four hours. After stirring at room temperature for 12 h, the solid was filtered and freeze-dried on vacuum. The dried solid was recrystallized from 10-12 parts of hot water to give compound 23b (17.9 g, 61%) as white needle crystals. Mp: 204-206° C. (H₂O); ¹H NMR (⁴d-MeOH, 300 MHz): δ 1.16 (d, J=6.6 Hz, 3H), 1.44 (s, 9H), 1.81-1.90 (m, 2H), 2.46-2.48 (m, 1H), 4.06 (dd, 1H); MS: m/e 282.1 (M⁺+23).

Method B: To a solution of compound 3b (11.6 g, 40.1 mmol) in THF (60 mL) was added dropwise aqueous 1 N NaOH (90 mL) at −10° C. to −5° C. with stirring. The stirring was continued for one hour at 0-5° C. and checked by LC/MS. At the end of the reaction (˜one hour), the reaction was treated with 3 N HCl (35-40 mL) until the color changed to Congo red. The aqueous solution was extracted with ethyl acetate (160 mL×2). The combined extracts were evaporated under reduced pressure to give (2S,45)-2-tert-butoxycarbonylamino-4-methyl-pentanedioic acid (compound 26b) (12.5 g, ˜100% crude yield, vacuum dry) as viscous oil. ¹H NMR (CDCl₃, 300 MHz): δ 1.22 (d, J=7.0 Hz, 3H), 1.43 (s, 9H), 2.02-2.07 (m, 1H), 2.28 (ddd, 1H), 2.62-2.68 (m, 1H), 4.50 (m, 1H), 5.26 (d, J=6.6 Hz, 1H); MS: m/e 284.0 (M⁺+23).

A solution of compound 26b (12.5 g) in THF (116 mL) was added in successively pyridine (3.9 g, 49.3 mmol), Boc₂O (23.5 g, 107.7 mmol) and ammonium bicarbonate (8.1 g, 102.5 mmol) with stirring. The reaction turns gradually from clear into a white powder suspension. After stirring for 12 hours at room temperature, the precipitate was filtered off and dried on vacuum to give compound 23b (10.3 g, 99%). HPLC analysis reveals that the purity of compound 23b is 95.2% and the de value of compound 23b was 99.4%.

### Example 11

### Synthesis of (2S,45)-2-tent-butoxycarbonylamino-4-methyl-pentanedioic acid (compound 26b) from y-methyl (2R)-N-Boc-L-glutamate and conversion of 26b to diamide (compound 23b)

Method A: A solution of diisopropylamine (5.3 g, 52.4 mmol) in 40 mL THF was cooled to −70° C., and n-butyllithium (21 mL, 2.5 M in hexane) was added via a cannula at the temperature <−60° C. The yellow clear solution was stirred at −70° C. for 0.5 h and 0° C. for 15 minutes. The dried lithium salt of y-methyl (2R)-N-Boc-L-glutamate (5.5 g, 20.6 mmol, prepared by titration of 5.4 g free acid to pH 8.0) in THF (27 mL) was added at −60 to −70° C. during a 40-minute period, and the thus-obtained cloudy mixture was diluted with 5-10 mL THF under vigorous stirring. MeI (4.6 g, 32.4 mmol) in THF (10 mL) was syringed in during a 15-minute period at the temperature of −60 to −70° C. After stirring for 1 h, additional MeI (1.0 g) was syringed in. The reaction was stirred at −70 to −30° C. for 1 h and maintained at −30° C. until LC/MS showed a major signal of lactam 26b″. The resulting mixture was acidified to pH 1 to 2 with 6 N HCl at <−10° C. and diluted with toluene (50 mL) under stirring. The organic phase was washed successively with a Na₂S₂O₃ solution (1.5 g in 20 mL water) and water (50 mL). It was evaporated to give lactam 26b″ (4.4 g, 88%). MS: m/e 266.0 (M⁺+23). Dicyclohexylamine (DCHA) salt (26b″•DCHA): MP 162-164° C. (t-BuOMe). To an ice-cooled solution of 26b″ (4.4 g) in THF (25 mL) was added an ice-cooled solution of lithium hydroxide monohydrate (2.0 g) in water (18 mL). After stirring at 0° C. for 3 h, the resulting mixture was acidified to pH 1 to 2 with 6 N HCl at <−10° C. and diluted with ethyl acetate (30 mL) also under stirring. The organic phase was washed with water (30 mL) and evaporated to give 26b (4.1 g, 76% based on y-methyl-N-Boc-L-glutamate). The diamide 23b (2.6 g, 49% based on y-methyl-N-Boc-L-glutamate) was prepared from 26b (4.1 g, 15.7 mmol) in a manner similar to Method B described in Example 10. HPLC analysis shows that the de value of compound 23b was 95%.

Method B (one-pot from y-methyl (2R)-N-Boc-L-glutamate): A solution of diisopropylamine (9.2 g, 90.9 mmol) in 80 mL THF was cooled to −70° C., and n-butyllithium (36.4 mL, 2.5 M in hexane) was added via a cannula at the temperature <−60° C. The yellow clear solution was stirred at −70° C. for 0.5 h and 0° C. for 15 minutes. The dried lithium salt of y-methyl (2R)-N-Boc-glutamate ester (11.0 g, 41.2 mmol), prepared by titration of 10.8 g free acid to pH 8.0) in THF (60 mL) was added at −60 to −70° C. over a 40-minute period, and the thus-obtained cloudy mixture was diluted with 5-10 mL THF under vigorous stirring. MeI (10.2 g, 71.9 mmol) in THF (15 mL) was syringed in at the temperature of −60 to −70° C. over a 15-minute period. The reaction was then stirred at −70° C. for 3.5 h. Subsequently a sodium hydroxide aqueous solution (1 N, 42 mL) was added at −30° C. and then stirred at 0° C. for 3 h. The resulting mixture was acidified to pH 1 to 2 with 6 N HCl at <−10° C. and diluted with ethyl acetate (100 mL) under stirring. The organic phase was washed with water (50 mL) and evaporated to give diacid 26b (10.7 g, 99%). Diamide 23b (5.2 g, 49% based on y-methyl (2R)-N-Boc-glutamate ester) was prepared from 26b (10.7 g) in a manner similar to Method B described in Example 10. HPLC analysis shows that the de value of compound 23b was 94%.

### Example 12

### Synthesis of (2S,45)-2-tent-butoxycarbonylamino-4-methyl-pentanedinitrile (compound 24b)

Method A: To an ice-cooled suspension of compound 23b (12.3 g, 47.4 mmol) in dichloromethane (70 mL) containing pyridine (23.5 g, 297.1 mmol) was added dropwise benzenesulfonyl chloride (31.1 g, 176.1 mmol). After the addition, the ice bath was removed and the reaction was allowed to stir at room temperature for 30 hours. The mixture was then diluted with dichloromethane (70 mL) and washed with water (70 mL×2). The separated organic layer was evaporated and the residue filtered through a plug of 5 parts of silica gel eluted with 2/3 (v/v) ethyl acetate-hexane. The collected solution was evaporated. The residual solid was recrystallized from hot 1/1 (v/v) t-BuOMe-hexanes (118 mL) to give compound 24b (9.7 g, 92%) as white crystals. Alternatively, the crude residue was directly purified by recrystallization from 4-5 parts of hot 1/1 (v/v) t-BuOMe-hexanes in 86% isolated yield. GC analysis reveals that the purity of compound 24b is 93% and the de value of compound 24b is greater than 99%. Mp: 108-110° C. (1/1 t-BuOMe-hexanes); ¹H NMR (CDCl₃, 300 MHz): δ 1.40 (d, J=7.2 Hz, 3H), 1.45 (s, 9H), 2.05-2.17 (m, 2H), 2.79-2.82 (m, 1H), 4.70 (br s, 1H), 5.00 (br d, J=8.7 Hz,1H); MS: m/e 246.0 (M^{|}+23).

Method B: To an ice-cooled solution of compound 23b (181.0 g, 698.8 mmol) in DMF (905 mL) was added cyanuric chloride (128.8 g, 698.4 mmol) in one-portion at 0-10° C. After being stirred for 1.5 h at 0-10° C., the ice bath was removed and the stirring was continued for 2.5 hour at ambient temperature. The mixture was then poured into ice water (2.5 L) during a period of 5 minutes with stirring; and then stirred for 10 minutes to allow white solid to precipitate out as needles. The slurry was filtered and the solid was washed with water (500 mL) to give crude compound 24b (160.0 g, >99%) after drying. The crude compound was dissolved in 800 mL (˜5 parts) of hot ethyl acetate (50-60° C.) and filtered through Celite to remove insoluble solids. The filtrate was evaporated under vacuum to yield compound 24b (125.0 g, 80%). GC analysis reveals that the purity of 24b is 93% and the de value of 24b is greater than 99%.

Method C (one-pot from compound 26b): To a solution of compound 26b (21 g) in DMF (147 mL) was added in successively Boc₂O (45.0 g, 206.2 mmol), ammonium bicarbonate (15.7 g, 198.6 mmol) and pyridine (7.6 g, 96.1 mmol) with stirring. The reaction turns gradually from clear into a white powder suspension. After stirring for 4 hours at room temperature, the mixture was evaporated by a rotary at below 45° C. to remove volatiles. The resulting solution was cooled in an ice-bath, and then treated with cyanuric chloride (14.8 g, 80.3 mmol) in one-portion at 0-10° C. After being stirred for 2.0 h at ice-bath temperature, additional amounts of cyanuric chloride (7.4 g) and DMF (40 mL) were added and stirring was continued for 1.5 h at ambient temperature. The mixture was poured into ice water (560 mL) during a period of 5 minutes with stirring; and then stirred for ten minutes to allow white solid to precipitate out as needles. The slurry was filtered and the solid was washed in successive with water (500 mL) to give crude compound 24b (23.0 g, >99%) after drying. The crude 24b was dissolved in 115 mL (˜5 parts) of hot ethyl acetate (50-60° C.) and filtered through a short pad of silica gel to remove insoluble solids. The filtrate was evaporated under vacuum to yield compound 24b (11.5 g, 64% based on compound 26b). GC analysis reveals that the purity of compound 24b is 93% and the de value is greater than 99%.

### Example 13

### Synthesis of compound 6b by reductive amination of (S)-2-tert-butoxycarbonylamino-pentanedinitrile (compound 24b) by catalytic hydrogenation

Method A: To a solution of compound 24b (3.6 g, 16.1 mmol) in MeOH (120 mL) containing Raney Nickel (˜3 g, wet weight) was added aqueous ammonia (24 mL, 28-32%). The mixture was then hydrogenated on a Parr Shaker under 80 psi pressures, and monitored by LC/MS. At the end of the reaction, the mixture was filtered from Celite and evaporated. The residual oil was filtered from a plug of silica gel (5-10 parts) using ethyl acetate containing 0.1% triethylamine and, then, evaporated to give compound 6b (2.4 g, 69%).

Compound 6b was also isolated in salt form. The hydrogenated solution was filtered from Clay (activated, 100 mesh), evaporated and the residue was dissolved in 10 parts hot i-PrOH. The resulting solution was treated with 0.5-0.6 molar equivalent of oxalic acid with heating to clearness, and then stood at room temperature overnight. Compound 6b•0.5 H₂C₂O₄ (2.3 g, 55%) was isolated as a white powder in successively by filtration and trituation over t-BuOMe and THF. GC analysis reveals that the de value of this compound is 94%.

Compound 6b was also prepared using 1/20-1/5 (v/v) ammonia water-methanol and/or ammonium salt additive, or using 10% Pd—C as the catalyst in the similar manners to that described above.

Method B: A solution of compound 24b (8.4 g, 37.6 mmol) and benzylamine (6.0 g, 56.1 mmol, 1.5 molar equivalent) in MeOH (240 mL) containing 10% Pd—C (4.2 g) was hydrogenated on a Parr Shaker under 80 psi pressures, and monitored by LC/MS. At the end of the reaction, the mixture was filtered from a short pad of Clay (activated, 100 mesh) and evaporated to give compound 6b (8.0 g, 99%). Compound 6b was also prepared using less than 1.5 molar equivalent of benzylamine (i.e. 1.0 to 1.5 molar equivalents) in about the same yield. The ¹H-NMR of the compound 6b was found identical to that of authentic sample, and a clean doublet appeared at 0.8 ppm confirms the desired stereochemistry with lack of existence of stereoisomeric side products. Crystallization of the crude 6b from 4-5 parts of n-heptane at −5° C. gave pure compound 6b as a colorless granular crystal. GC analysis reveals that the de value of this compound is 94%.

Compound 6b had an optical purity greater than 98% determined by the chiral urea method described in Example 7.

Method C: A solution of compound 24b (5.0 g, 22.4 mmol) and benzylamine (2.5 g, 23.3 mmol, 1.04 molar equivalent) in 7 N methanolic ammonia (50 mL, 15.6 molar equivalent) containing 10% Pd—C (1.5 g, containing 50 wt% water, Aldrich Degussa type) and activated charcoal (0.5 g) was hydrogenated on a Parr Shaker under 80 psi pressures. The hydrogenation reaction was monitored by LC/MS. At the end of the reaction, the mixture was filtered through Celite and evaporated to give compound 6b (5.1 g, >99% crude yield). GC analysis showed that the compound contained 0.85% diastereoisomer impurity. To remove the diastereomer impurity, compound 6b was purified as follows:

To a suspension of oxalic acid (1.06 g, about 0.5 molar equivalent) in acetone (53 mL) and water (5.1 mL) was added a solution of the crude compound 6b (5.1 g) in acetone (53 mL) over a 5-minute period at 40° C. with vigorous stirring. The mixture was heated under reflux with vigorous stirring for 9 h. The re-cooled mixture was filtered and washed with cold acetone to give oxalate of compound 6b. To a suspension of the oxalate (4.8 g) in water (20.7 mL) was added in successive 10% Na₂CO₃ (27.0 g, 6.0 parts) and ethyl acetate (45 mL) with stirring. After 15 minutes of stirring, the resultant was filtered through a Sintered glass funnel to remove suspended sodium oxalates. The organic layer was collected and the aqueous layer was extracted with ethyl acetate (45 ml). The combined organic extracts (˜90 mL) was washed with water (18 mL), and evaporated to give pure compound 6b (3.6 g, 75%) as white powders. Mp: 63-64° C. (hexane). GC analysis reveals that the purity of 6b is greater than 99% and no diastereomer impurity exists in compound 6b.

Compound 6b was obtained at comparable yield and purity when a less amount (i.e., 5, 10, or 20 mL) of 7 N methanolic ammonia was added. For each run, the total volume of the solvent was kept at 50 mL. Compound 6b exhibited an optical purity greater than 99%, which was determined by the method described in Example 7.

### Example 14

### Resolution of compound 6b

To remove stereoisomers, crude compound 6b was purified by recrystalization with 0.5 molar equivalent of D-(−)-tartaric acid in hot acetone-water (18/1 (v/v) to 36/1 (v/v)). Purified compound 6b (73% recovery) had greater than 99.5% desired isomer purity determined by GC analysis. Other chiral acids, such as (+)-dibenzoyl-D-tartaric acid and (+)-di-1,4-toluoyl-D-tartaric acid, were also employed to improve the isomer purity with acceptable recovery.

Compound 6b exhibited an optical purity greater than 99.5%, which was determined by the chiral urea method described in Example 7.

### Example 15

### Synthesis of (25)-2-tert-Butoxycarbonylamino-pentanedioic acid diamide (compound 27b)

A solution of N-Boc-L-glutamic acid (38.7 g, 156.5 mmol) in THF (450 mL) was added to successively pyridine (15.1 g, 190.9 mmol), Boc₂O (91.2 g, 417.9 mmol), and ammonium bicarbonate (31.4 g, 397.2 mmol) with stirring. After 12 hours at room temperature, the reaction mixture was evaporated. The residue was triturated over t-BuOMe (500 mL) and the precipitate was collected by filtration and dried under vacuum to give compound 27b (37.3 g, 97%). MP: 130-132° C. (MeOH); ¹H NMR (⁴d-MeOH, 300 MHz): δ1.45 (s, 9H), 1.85-1.90 (m, 1H), 2.04-2.07 (m, 1H), 2.31 (t, J=7.7 Hz, 2H), 4.02-4.05 (m, 1H); MS: m/e 268.1 (M⁺+23).

### Example 16

### Synthesis of (25)-2-tert-butoxycarbonylamino-pentanedinitrile (compound 28b)

To an ice-cold solution of 27b (37.0 g, 150.9 mmol) in DMF (185 mL) was added cyanuric chloride (27.7 g, 150.2 mmol) in one-portion at 0-10° C. After being stirred for 1.5 h at the same temperature, the ice bath was removed and stirring was continued for 1.5 h at ambient temperature. The mixture was then poured into ice water (555 mL) during a period of five minutes with stirring; and then stirred for ten minutes to give a slurry. The slurry was filtered and the solid was washed with water (200 mL) and dried. The filtrate was extracted with ethyl acetate (200 mL). Dissolved the solids in the extract and filtered through a short pad of silica gel. The filtrate was evaporated and the residual solid was dissolved in 110 mL of hot t-BuOMe (60° C.) and then diluted with hexane (200 mL). After 3 h at room temperature, the mixture was filtered and washed with 50 mL of hexane to give 28b (26.0 g, 82%) as white powder crystals. MP: 94-96° C. (hexane); ¹H NMR (CDCl₃, 300 MHz): δ1.45 (s, 9H), 2.10-2.20 (m, 2H), 2.40-2.60 (m, 2H), 4.60-4.70 (m, 1H), 5.00-5.20 (m, 1H); MS: m/e 232.1 (M⁺+23).

The title compound 28b (25.5 g, 81%) was also prepared from 27b (37.0 g, 0.15 mol) in a manner similar to Method A described in Example 12.

### Example 17

### Synthesis of (2S, 4S)-2-tert-butoxycarbonylamino-4-methyl-pentanedinitrile (compound 24b) and conversion to compound 6b

1 M LiHMDS in THF (110 mL) was charged into a 500 mL flask at −78° C. under nitrogen. To this solution was added dropwise a solution containing compound 28b (10.5 g, 50.0 mmol, in 80 mL dry THF) below −65° C., and then stirred for 3 h at −78° C. To the resulting solution MeI was added (4.7 mL) below −65° C. The reaction was stirred at −65 to −78° C. and monitored by LC/MS. After 3 h, the reaction was quenched with MeOH (2.4 mL), at −60° C., and 2 N HCl (167 mL) at −10° C. Toluene (70 mL) was added and the mixture was stirred for 0.5 h. The organic layer was separated and treated with a Na₂S₂O₃ solution (11 g in 96 mL water) with stirring for 30 minutes. The organic layer was evaporated under vacuum to give a crude product, which was purified by recrystallization using 1/5 (v/v) tert-BuOMe-hexane to yield compound 24b (9.3 g, 83%). GC analysis reveals that the ratio of compound 24b and its diastereomer is 2:1. After catalytic hydrogenation of compound 24b and the oxalate triturating purification (Table 1), compound 6b having a 96% de value (3.3 g, 31% based on 28b) was obtained.

### Example 18

### Synthesis of (3S,5S)-3-(tent-butoxycarbonylamino)-5-methylpiperidine (compound 6b)

The title compound 6b (9.5 g, 73%) was prepared from 24b (13.6 g, 60.9 mmol) in a manner similar to Method C described in Example 13. GC analysis revealed that compound 6b had a de value of 94%.

### Example 19

### Synthesis of (2S)-2-tert-butoxycarbonylamino-butanedioic acid diamide (compound 29b)

To a solution of N-Boc-L-aspartic acid (29.5 g, 126.5 mmol) in THF (348 mL) was added pyridine (11.7 g, 147.9 mmol), Boc₂O (70.5 g, 323.0 mmol), and ammonium bicarbonate (24.3 g, 307.4 mmol) with stirring. The reaction mixture was stirred for 12 hours at room temperature and then evaporated. The residue was diluted with ethyl acetate (250 mL) and washed with water (50 mL) with stirring. The organic layer was evaporated to give compound 29b (20.1 g, 69%). MP: 190-192° C. (MeOH); ¹H NMR (⁴d-MeOH, 300 MHz): δ1.44 (s, 9H), 2.64, 2.59 (ABq, J=5.4 Hz, 2H), 4.37-4.45 (m, 1H); MS: m/e 254.1 (M⁺+23).

### Example 20

### Synthesis of (2S)-2-tent-butoxycarbonylamino-butanedinitrile (compound 28a)

Compound 28a (9.0 g, 54%) was prepared from compound 27a (19.8 g, 85.6 mmol), pyridine (45.0 g, 6.65 eq), and benzenesulfonyl chloride (59.6 g, 3.9 eq) in a manner similar to Method A described in Example 12. MP: 134-136° C. (hexane); ¹H NMR (CDCl₃, 300 MHz): δ1.46 (s, 9H), 2.95 (dd, J=6.6, 3.4 Hz, 2H), 4.80-4.96 (m, 1H), 5.32 (d, J=8.7 Hz, 1H),; MS: m/e 218.0 (M⁺+23).

### Example 21

### Synthesis of (3S)-3-(tent-butoxycarbonylamino)-pyrrolidine (compound 29a)

Compound 29a (7.0 g, 71%) was prepared from 28a (10.3 g, 52.8 mmol) in a manner similar to Method C described in Example 13.

Compound 29a•0.5 H₂C₂O₄, MP: 170° C. (dec.) (t-BuOMe); ¹H NMR (⁴d-MeOH, 300 MHz): δ1.44 (s, 9H), 1.90-2.10 (m, 1H), 2.20-2.30 (m, 1H), 3.17-3.37 (m, 2H), 3.38-3.45 (m, 2H), 4.20-4.24 (m, 1H); MS: m/e 187.1 (M⁺+1).

### Example 22

### Synthesis of (3S)-3-(tent-butoxycarbonylamino)-piperidine (compound 29b)

Compound 29b (7.0 g, 72%) was prepared from 28b (10.1 g, 48.3 mmol) in a manner similar to Method C described in Example 13. Compound 29b had an optical purity greater than 99% determined by the method described in Example 7.

### Example 23

### Synthesis of 2-tert-butoxycarbonylamino-hexanedioic acid diamide (compound 27c)

Compound 27c (2.4 g, 76%) was prepared from 2-tert-butoxycarbonylamino-pentanedioic acid (3.2 g, 12.3 mmol) by in a manner similar to Method B described in Example 10. MP: 135-137° C. (MeOH); ¹H NMR (⁴d-MeOH, 300 MHz): δ1.43 (s, 9H), 1.60-1.66 (m, 2H), 1.70-1.77 (m, 2H), 2.25 (t, J=5.4 Hz, 2H), 3.98-4.02 (m, 1H); MS: m/e 282.0 (M⁺+23).

### Example 24

### Synthesis of 2-tert-butoxycarbonylamino-hexanedinitrile (compound 28c)

Compound 28c (1.5 g, 73%) was prepared from 27c (2.4 g, 9.3 mmol) in a manner similar to Method B described in Example 12. MP: 61-63° C. (hexane); ¹H NMR (CDCl₃, 300 MHz): δ 1.44 (s, 9H), 1.80-1.87 (m, 2H), 1.90-2.00 (m, 2H), 2.43 (t, J=6.6 Hz, 2H), 4.50-4.60 (m, 1H), 5.00-5.20 (m, 1H); MS: m/e 246.0 (M⁺+23).

### Example 25

### Synthesis of 3-tert-butoxycarbonylaminohexahydro-2-azepine (compound 29c)

Compound 29c (3.4 g, 71%) was prepared as yellow oil from compound 28c (5.0 g, 22.4 mmol) in a manner similar to Method C described in Example 13. ¹H NMR (CDCl₃, 300 MHz): δ 1.41 (s, 9H), 1.55-1.62 (m, 4H), 1.65-1.80 (m, 2.73, 2.78 (ABq, J=4.8 Hz, 2H), 2.87, 2.91 (ABq, J=3.6 Hz, 2H), 3.60-3.70 (m, 1H), 5.00-5.10 (m, 1H); MS: m/e 215.1 (M⁺+1). Compound 29c•oxalate: MP: 207° C. (dec.) (t-BuOMe).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula I: 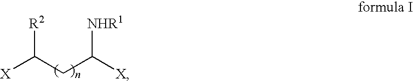
wherein R¹ is an amino-protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; X is C(O)H or CN; and n is 0, 1, or 2.

2. The compound of claim 1, wherein R² is C₁-C₆ alkyl.

3. The compound of claim 2, wherein R¹ is C(O)Ot-Bu, C(O)OCH₂Ph, C(O)CH₃, C(O)CF₃, CH₂Ph, or C(O)O-Ph.

4. The compound of claim 1, wherein X is C(O)H.

5. The compound of claim 4, wherein the compound is

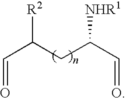

6. The compound of claim 5, wherein R¹ is C(O)Ot-Bu, C(O)OCH₂Ph, C(O)CH₃, C(O)CF₃, CH₂Ph, or C(O)O-Ph; R² is C₁-C₆ alkyl; and n is 0, 1, or 2.

7. The compound of claim 4, wherein the compound is

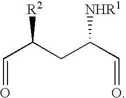

8. The compound of claim 7, wherein R¹ is C(O)Ot-Bu, C(O)OCH₂Ph, C(O)CH₃, C(O)CF₃, CH₂Ph, or C(O)O-Ph and R² is C₁-C₆ alkyl.

9. The compound of claim 1, wherein X is CN.

10. The compound of claim 9, wherein the compound is

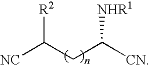

11. The compound of claim 10, wherein R¹ is C(O)Ot-Bu, C(O)OCH₂Ph, C(O)CH₃, C(O)CF₃, CH₂Ph, or C(O)O-Ph; R² is H or C₁-C₆ alkyl; and n is 0, 1, or 2.

12. The compound of claim 9, wherein the compound is

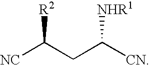

13. The compound of claim 12, wherein R¹ is C(O)Ot-Bu, C(O)OCH₂Ph, C(O)CH₃, C(O)CF₃, CH₂Ph, or C(O)O-Ph and R² is C₁-C₆ alkyl.

14. The compound of claim 1, wherein the compound is

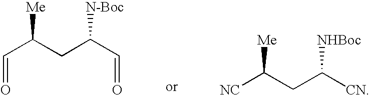

15. A synthetic process comprising:
conducting a cyclization reaction by contacting a compound of formula I: 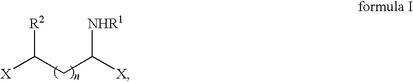
wherein R¹ is an amino-protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; X is C(O)H or CN; and n is 0, 1, or 2;
with a compound of formula II:
H₂NR³ formula II,
wherein R³ is H, C₁-C₆ alkyl, C₂-C₆ alkenyl, C₂-C₆ alkynyl, C₃-C₈ cycloalkyl, C₁-C₇ heterocycloalkyl, aryl, heteroaryl, to form a compound of formula III: 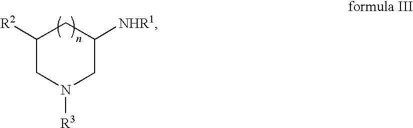
in which R¹, R², R³ and n are as defined above.

16. The process of claim 15, wherein R¹ is C(O)Ot-Bu, C(O)OCH₂Ph, C(O)CH₃, C(O)CF₃, CH₂Ph, or C(O)O-Ph; R² is H or C₁-C₆ alkyl; R³ is H or CH₂Ph; and n is 1.

17. The process of claim 16, further comprising removing R³ from the compound of formula III and coupling the resultant compound with a quinolinone compound to form a compound of the following formula: 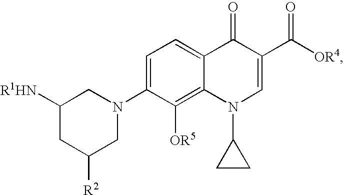
wherein R¹ is H, C(O)Ot-Bu, C(O)OCH₂Ph, C(O)CH₃, C(O)CF₃, CH₂Ph, or C(O)O-Ph; R² is H or C₁-C₆ alkyl; R⁴ is H or carboxyl protecting group; and R⁵ is H, C₁-C₆ alkyl, C₂-C₆ alkenyl, C₂-C₆ alkynyl, C₃-C₈ cycloalkyl, C₁-C₇ heterocycloalkyl, aryl, or heteroaryl.

18. The process of claim 15, wherein the compound of formula I is 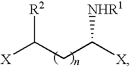
and the compound of formula III is

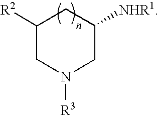

19. The process of claim 18, wherein R¹ is C(O)Ot-Bu, C(O)OCH₂Ph, C(O)CH₃, C(O)CF₃, CH₂Ph, or C(O)O-Ph; R² is H or C₁-C₆ alkyl; R³ is H or CH₂Ph; and n is 1.

20. The process of claim 19, further comprising removing R³ from the compound of formula III and coupling the resultant compound with a quinolinone compound to form a compound of the following formula: 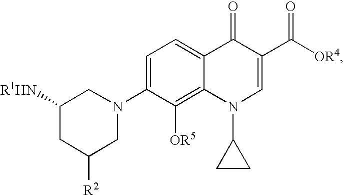
wherein R¹ is H, C(O)Ot-Bu, C(O)OCH₂Ph, C(O)CH₃, C(O)CF₃, CH₂Ph, or C(O)O-Ph; R² is H or C₁-C₆ alkyl; R³ is H or CH₂Ph; R⁴ is H or carboxyl protecting group; and R⁵ is H, C₁-C₆ alkyl, C₂-C₆ alkenyl, C₂-C₆ alkynyl, C₃-C₈ cycloalkyl, C₁-C₇ heterocycloalkyl, aryl, or heteroaryl.

21. The process of claim 15, wherein the compound of formula I is 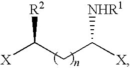
and the compound of formula III is

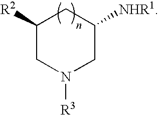

22. The process of claim 21, wherein R¹ is C(O)Ot-Bu, C(O)OCH₂Ph, C(O)CH₃, C(O)CF₃, CH₂Ph, or C(O)O-Ph; R² is C₁-C₆ alkyl; R³ is H or CH₂Ph; and n is 1.

23. The process of claim 22, further comprising removing R³ from the compound of formula III and coupling the resultant compound with a quinolinone compound to form a compound of the following formula: 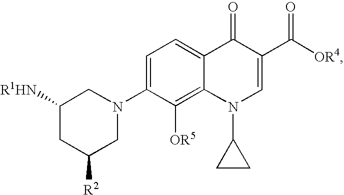
wherein R¹ is H, C(O)Ot-Bu, C(O)OCH₂Ph, C(O)CH₃, C(O)CF₃, CH₂Ph, or C(O)O-Ph; R² is H or C₁-C₆ alkyl; R³ is H or CH₂Ph; R⁴ is H or carboxyl protecting group; and R⁵ is H, C₁-C₆ alkyl, C₂-C₆ alkenyl, C₂-C₆ alkynyl, C₃-C₈ cycloalkyl, C₁-C₇ heterocycloalkyl, aryl, or heteroaryl.

24. The process of claim 23, wherein R¹ is H, R² is CH₃, R⁴ is H, and R⁵ is CH₃.

25. The process of claim 15, wherein the compound of formula I is an dialdehyde compound as shown below:

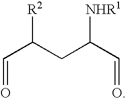

26. The process of claim 25, further comprising, before the cyclization reaction, obtaining the dialdehyde compound by conducting a reduction reaction of a compound of formula IV, 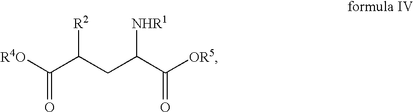
in which R¹ is an amino protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; and each of R⁴ and R⁵, independently, is H, C₁-C₆ alkyl, C₂-C₆ alkenyl, C₂-C₆ alkynyl, C₃-C₈ cycloalkyl, C₁-C₇ heterocycloalkyl, aryl, or heteroaryl.

27. The process of claim 26, wherein the aldehyde compound is 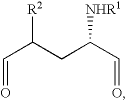
the compound of formula IV is 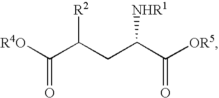
and the compound of formula III is

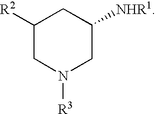

28. The process of claim 27, wherein the aldehyde compound is 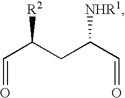
the compound of formula IV is 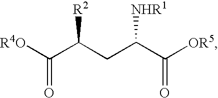
and the compound of formula III is:

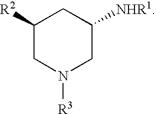

29. The process of claim 26, wherein the reduction reaction and the cyclization reaction are performed in a one-pot fashion.

30. The process of claim 25, further comprising, before the cyclization reaction, reducing a compound of formula IV: 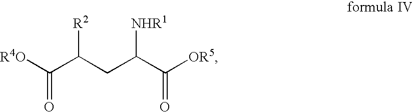
in which R¹ is an amino protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; and each of R⁴ and R⁵, independently, is C₁-C₆ alkyl, C₂-C₆ alkenyl, C₂-C₆ alkynyl, C₃-C₈ cycloalkyl, C₁-C₇ heterocycloalkyl, aryl, or heteroaryl;
to form a dialcohol compound as shown below: 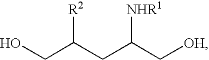
and oxidizing the dialcohol compound to provide the dialdehyde compound.

31. The process of claim 15, wherein each of X groups is CN.

32. The process of claim 31, further comprising, before the cyclization reaction, treating, with a dehydrating agent, a compound of formula V, 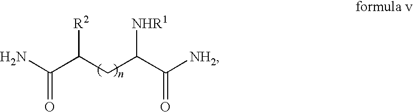
in which R¹ is an amino protecting group; R² is H, C₁-C₆ alkyl, C₂-C₆ alkenyl, C₂-C₆ alkynyl, C₃-C₈ cycloalkyl, C₁-C₇ heterocycloalkyl, aryl, or heteroaryl; and n is 0, 1, or 2; to provide the dinitrile compound.

33. The process of claim 32, wherein the dinitrile compound is 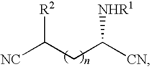
the compound of formula V is 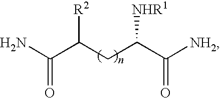
and the compound of formula III is

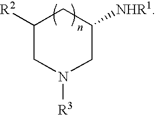

34. The process of claim 33, wherein the dinitrile compound is 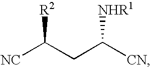
the compound of formula V is 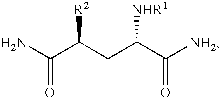
and the compound of formula III is

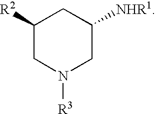

35. The process of claim 15, wherein the compound of formula I is 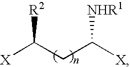
wherein n is 0, 1, or 2, R¹ is an amino protecting group, R² is alkyl, and each of X is CN.

36. The process of claim 35, further comprising treating the following compound 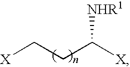
wherein n is 0, 1, or 2, R¹ is an amino protecting group, and each of X is CN, in the presence of a base, with R²L, wherein R² is alkyl and L is I, Br, or MeSO₄, to stereoselectively synthesize the compound of formula I.

37. The process of claim 36, wherein R² is methyl and the base is LiHMDS.

38. The process of claim 35, further comprising reacting the compound of formula III, wherein R³ is H, with an acid to form a salt and stereoselectively purifying the salt.

39. The process of claim 38, wherein the acid is oxalic acid or a chiral acid.
